(19) United States
(12) Patent Application Publication
(10) Pub. No.: US 2023/0113738 A1
(43) Pub. Date: Apr. 13, 2023

(54) FATTY ACID COMPOSITIONS FOR ENTERAL USE

(71) Applicant: OPTIMEGA LLC, Princeton, NJ (US)

(72) Inventor: Shaohui LIN, Princeton, NJ (US)

(21) Appl. No.: 17/905,676

(22) PCT Filed: Mar. 6, 2020
(86) PCT No.: PCT/US2020/021475

§ 371 Date: Sep. 6, 2022

(51) Int. Cl. A61K 9/107; A61K 35/60; A23L 33/12; A61K 47/14; A61K 9/00

(52) U.S. Cl. CPC A61K 9/107; A61K 35/60; A23L 33/12; A61K 47/14; A61K 9/0053

(57) ABSTRACT

This disclosure provides an isotonic lipid emulsion for enteral administration comprising Omega-3 fatty acids and medium-chain triglyceride (MCT) fatty acids. The lipid particles administered through the disclosed lipid emulsion can be efficiently absorbed by animal and human body and rapidly enriched on organ and tissue cell membranes.

### FIELD OF THE INVENTION

The present invention relates generally to compositions of fatty acids and more specifically fatty acid compositions that can be rapidly enriched on cell membranes for modifying the fatty acid composition of organ and tissue cell membranes.

### BACKGROUND OF THE INVENTION

Currently, most of the oral Omega-3 fatty acids commercially available are in the form of oily liquids, in which the Omega-3 long-chain unsaturated fatty acid triglycerides are usually esterified or ethylated. The oily liquids enter the intestine and are emulsified by bile salts, forming micelles with larger particle sizes. At the same time, the triglyceride fats are hydrolyzed by lipase into component fatty acid and glycerol molecules, and the small intestinal mucosa absorbs the hydrolyzed fatty acid fragments and combines cholesterol with the action of ApoC-II apolipoprotein to form chylomicrons. Chylomicrons are transported into tissues through the lymphatic system and blood. Lipoprotein lipase activates the ApoC-II apolipoproteins in capillaries to release free fatty acids, which enter cells to produce energy or participate in the construction of cellular structures. This lipid hydrolysis step has been considered to be a rate-limiting factor in lipid metabolism, which is undertaken by the relatively restricted activity of lipoprotein lipase in the decomposition of triglycerides.

U.S. patent publication US20170304249A1A discloses a concentrated emulsion and emulsion comprising Omega-3 fatty acids and describes the results of an emulsion and/or emulsion pre-concentrate in plasma compared to Omega-3 in oil form. It shows increased oral absorption (exposure) and improved oral bioavailability and treatment. For example, in the treatment of hypertriglyceridemia, the emulsion and/or emulsion pre-concentrate is superior to Omega-3 in oil form. However, the experimental data was based on the comparison of fatty acids in plasma, and the fatty acid index in plasma cannot reflect the degree of Omega-3 fatty acid enrichment on the cell membrane of organs and tissues. Thus, the testing of fatty acids on the blood cell membrane can more accurately reflect the degree of enrichment of fatty acids on the cell membrane of organs and tissues than testing the fatty acid content in plasma.

U.S. Pat. No. 4,871,768 describes a structured lipid compound containing Omega-3 fatty acids and medium-chain fatty acids. The patent recognizes that long-chain fatty acids are absorbed relatively slowly by the human body because they require initial hydrolysis and traveling through the lymphatic system. The patent claims that this structured lipid compound containing Omega-3 fatty acids and medium-chain fatty acids can provide high calories, as well as allowing Omega-3 fatty acids to be absorbed while avoiding damage to the reticuloendothelial system.

Enteral nutrition is an important means of nutritional support in a clinical context, which is more critical than parenteral nutrition. The importance of Omega-3 fatty acid—an essential fatty acid that cannot be synthesized by the human body—is self-evident and cannot be missed in clinical nutritional therapy. But the hurdles for enteral administration of Omega-3 fatty acids still exist in order to quickly provide Omega-3 fatty acids for people with Omega-3 fatty acid-deficiency, including bad tastes and the low bioavailability of Omega-3 fatty acid in the human body

To date, no isotonic lipid emulsion for enteral administration containing omega-3 fatty acids and MCT fatty acids has been reported or characterized. Accordingly, there is a strong need for an isotonic lipid emulsion suitable for enteral administration, especially the isotonic lipid emulsion with the ability to provide rapid enrichment of Omega-3 fatty acids on cell membranes.

### SUMMARY OF THE INVENTION

This disclosure addresses the need mentioned above in a number of aspects. In one aspect, this disclosure provides an isotonic lipid emulsion. The isotonic lipid emulsion comprises (i) 1% to 78% by weight of medium-chain triglycerides (MCT) based on the total amount of lipid within the emulsion, and (ii) 22% to 99% by weight of fish oil or krill oil based on the total amount of lipid within the emulsion, wherein the fish oil is selected from natural fish oil, processed fish oil, purified fish oil concentrate, (re)esterified synthetic fish oil and mixtures thereof, and fish oil extracted from biologically-engineered microorganisms, wherein the average particle size of emulsion particles is between about 10 nm and about 250 nm.

In some embodiments, the isotonic lipid emulsion is formulated for enteral administration. In some embodiments, the isotonic lipid emulsion is formulated for enteral administration through an oral, nasal, or jejunal feeding tube.

In some embodiments, the average particle size of the emulsion particles is between about 10 nm and about 200 nm. In some embodiments, at least 50% of the emulsion particles have particle sizes of 230 nm or less. In some embodiments, at least 90% of the emulsion particles have particle sizes of 600 nm or less.

In some embodiments, the isotonic lipid emulsion is an oil-in-water emulsion. In some embodiments, the oil component in the isotonic lipid emulsion has a concentration of from 2 g/100 mL to about 20 g/100 mL.

In some embodiments, the isotonic lipid emulsion has a pH between about 3.0 and about 8.0. In some embodiments, the isotonic lipid emulsion has a pH between about 3.0 and about 4.5 when the F0 value of sterilization temperature and time is less than or equal to 1. In some embodiments, the isotonic lipid emulsion has a pH between about 6.5 and about 7.5 when the F0 value of sterilization temperature and time is greater than or equal to 8.

In some embodiments, the osmotic pressure of the isotonic lipid emulsion is between about 280 mmol/L and about 320 mmol/L.

In some embodiments, the isotonic lipid emulsion is prepared from a concentrated, hypertonic lipid emulsion by dilution.

In some embodiments, the total lipid content is between about 5% and about 60% by weight of the liquid emulsion. In some embodiments, the total lipid content is between about 10% and about 30% by weight of the liquid emulsion. In some embodiments, the MCT contains 6 to 14 carbon atoms. In some embodiments, the MCT contains at least about 90% caprylic acid (C8), capric acid (C10), or a combination thereof. In some embodiments, the MCT is obtained from a source selected from the group consisting of plant extract, animal extract, and synthetic fatty acid.

In some embodiments, the fish oil is based on fatty acid methyl ester of fish oil concentrate and contains between about 25% and about 95% by weight of eicosapentaenoic acid (EPA) based on the total weight of the fish oil. In some embodiments, the fish oil contains between about 12% and about 95% by weight of docosahexaenoic acid (DHA) based on the total weight of the fish oil.

In some embodiments, the isotonic lipid emulsion further comprises an additional agent selected from the group consisting of emulsifier, emulsifying aid, stabilizer, antioxidant, ion antagonism, defoaming agent, natural (or synthetic) flavoring, natural (or synthetic) fragrance, and osmolarity-balancing agent.

In another aspect, this disclosure also provides a composition comprising the isotonic lipid emulsion, as described above. Also provided is a pharmaceutical composition comprising the isotonic lipid emulsion described above and a pharmaceutically acceptable carrier.

Also within the scope of this disclosure is a food or drink additive comprising the isotonic lipid emulsion as described above. In some embodiments, the food or drink additive is formulated for food or drink selected from the group consisting of water, fruit juice, and vegetable juice, or for natural (or synthetic) flavoring and fragrance for preparing one or more foods.

In another aspect, this disclosure further provides a method of administering a lipid emulsion. The method comprises administering enterally a dose of the isotonic lipid emulsion, the composition, or the pharmaceutical composition, as described above, to a subject in need thereof. In some embodiments, the subject is a mammal, such as human.

In yet another aspect, this disclosure additionally provides a method for treating a human having Omega-3 fatty acid deficiency. The method comprises enterally administering to the human an effective dosage amount of the isotonic lipid emulsion, the composition, or the pharmaceutical composition, as described above, thereby increasing enrichment of Omega-3 fatty acids on the cell membrane of human tissue and organ by at least 10% compared to a predetermined reference value. In some embodiments, the enteral administration is carried out through an oral, nasal, or jejunal feeding tube.

In some embodiments, the organ is selected from the group consisting of heart, kidney, brain, liver, lung, and adipose tissue. In some embodiments, the tissue is selected from the group consisting of endothelium, white blood cell, platelet, and immune cell.

In some embodiments, the human has a condition selected from the group consisting of a systemic inflammatory response syndrome, a respiratory distress syndrome, a nutritional and/or dietary cause of liver disease, an iatrogenic cause of liver disease, a pathological cause of liver disease, an immune modulation, head trauma, postoperative surgical stress, a myocardial infarction, cystic fibrosis, and a combination thereof.

In some embodiments, the human is in need of rapidly supplementing Omega-3 fatty acids to improve metabolic syndrome, or to benefit from the efficacy of Omega-3 fatty acids in modulating inflammation, prevention of premature birth, myocardial ischemia or infarction, transient local cerebral ischemia or stroke, autoimmunity, and thrombotic diseases, organ transplantation, acute phase response, acute respiratory distress syndrome, inflammatory bowel syndrome, and hypertriglyceridemia.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIG. 1 is a graph showing the effect of high osmotic pressure microemulsions, isotonic lipid emulsions, and conventional oil on the degree of EPA enrichment in a zebrafish experiment. In particular, it shows the evolution of the percentage of EPA in the phospholipid fraction of whole body fish after 10 and 20 days of supplementation.

FIG. 2 is a graph showing the effect of high osmotic pressure microemulsions, isotonic lipid emulsions, and conventional oil liquids on the degree of DHA enrichment in a zebrafish experiment. In particular, it shows the evolution of the percentage of DHA in the phospholipid fraction of whole Body fish after 10 and 20 days of supplementation.

FIG. 3 is a graph showing the effect of different Omega-3 fatty acid/MCT ratio on the degree of EPA enrichment in a zebrafish experiment. In particular, it shows the evolution of the percentage of EPA in the phospholipid fraction of whole body fish after 10 and 20 days of supplementation.

FIG. 4 is a graph showing the effect of different Omega-3 fatty acid/MCT ratio on the degree of DHA enrichment in a zebrafish experiment. In particular, it shows the evolution of the percentage of DHA in the phospholipid fraction of whole Body fish after 10 and 20 days of supplementation.

FIG. 5 is a graph showing EPA enrichment on leukocyte phospholipid membrane of 43 volunteers taking lipid emulsion on day 0, day 2, day 4, day 7, day 10, day 14, day 21, day 28 in a human experiment.

FIG. 6 is a graph showing DHA enrichment on leukocyte phospholipid membrane of 43 volunteers taking lipid emulsion on day 0, day 2, day 4, day 7, day 10, day 14, day 21, day 28 in a human experiment.

FIG. 7 is a graph showing EPA enrichment on platelet phospholipid membrane of 43 volunteers taking lipid emulsion on day 0, day 2, day 4, day 7, day 10, day 14, day 21, day 28 in a human experiment.

FIG. 8 is a graph showing DHA enrichment on platelet phospholipid membrane of 43 volunteers taking lipid emulsion on day 0, day 2, day 4, day 7, day 10, day 14, day 21, day 28 in a human experiment.

FIGS. 9a and 9b (collectively “FIG. 9”) are a set of diagrams showing size distribution by intensity of lipid emulsion.

### DESCRIPTION OF THE INVENTION

The present invention is based, at least in part, on the unexpected discovery that when Omega-3 fatty acids and medium-chain triglyceride (MCT) fatty acids are combined in a particular ratio and enterally administered, they can be rapidly absorbed by animal and human body and rapidly enriched on the organ or tissue cell membranes (e.g., blood cell membranes). Also, the amount of Omega-3 fatty acid enrichment increases with an increased number of times of administration. Thus, the isotonic lipid emulsion, as disclosed herein, is suitable for increasing the amount of Omega-3 fatty acids on organ and tissue cell membranes by the form of isotonic lipid emulsion fatty acid comprising the selected fatty acid triglycerides.

### A. ISOTONIC LIPID EMULSION

The disclosed isotonic lipid emulsion has one or more of the following characteristics: (1) it has a particle size (e.g., mean particle size, average particle size, largest dimension) in the range of about 10 nm to about 900 nm; (2) it has a pH in the range of about 3.0 to about 8.0; (3) it has an osmotic pressure between about 280 mmol/L and about 320 mmol/L; (4) the lipid emulsion comprises, based on the total amount of lipid in the lipid emulsion, (i) about 1% to about 78% by weight of MCT, and (ii) about 22% to about 99% by weight of Omega-3 fatty acid; (5) it has a total lipid content between about 5% and about 60% by weight based on the total weight of the lipid emulsion; (6) it masks the fishy smell of the lipid by taste modifying and taste masking techniques, resulting in pleasant taste of the lipid emulsion; (7) it is a fatty acid composition comprising Omega-3 fatty acid that is formulated for enteral administration and can be rapidly absorbed by the cells and subsequently enriched on cell membranes. In addition, the lipid emulsion is capable of rapidly altering the fatty acid composition of organ and tissue cell membranes; and (8) it provides a rapid supplementation of Omega-3 fatty acids to a subject (e.g., human, animal) with Omega-3 fatty acid deficiency.

A lipid emulsion of the present invention is uniformly an oil-in-water (O/W) emulsion, which can be obtained by mixing Omega-3 fatty acid and medium-chain fatty acid, followed by emulsification and sterilization. A process of preparing the lipid emulsion includes first mixing lipids, emulsifiers, and other processing agents and additives, and then supplementing with water while dispersing. Water may optionally contain additional water-soluble particles (e.g., glycerol, PEG400). The lipid emulsion obtained may contain lipid particles with a diameter of approximately 10 microns, which can be further reduced by an additional homogenization operation. In some embodiments, an average emulsion particle size ranges from about 10 nm to about 900 nm, e.g., ranging from about 70 nm to about 500 nm.

In some embodiments, the disclosed isotonic lipid emulsion comprises: (i) 1% to 78% by weight of MCT based on the total amount of lipid within the emulsion, and (ii) 22% to 99% by weight of fish oil or krill oil based on the total amount of lipid within the emulsion. The fish oil can be selected from natural fish oil, processed fish oil, purified fish oil concentrate, (re)esterified synthetic fish oil and mixtures thereof, and fish oil extracted from biologically-engineered microorganisms, wherein the average particle size of emulsion particles is between about 10 nm and about 230 nm.

The isotonic lipid emulsion may further include one or more additional agents. For example, the lipid emulsion can be obtained by mixing lipids, emulsifiers, and other processing agents or additives. The step of mixing can be carried out in different ways, such as: (1) aqueous phase liquid containing emulsifiers, processing agents, and additives is added to oil phase liquid containing emulsifiers, processing agents and additives in proportions, followed by rapid stirring or shearing; or (2) oil phase liquid containing emulsifiers, processing agents and additives is added to aqueous phase liquid containing emulsifiers, processing agents and additives in proportions, following by rapid stirring and shearing; or (3) oil phase liquid (or aqueous phase liquid) containing emulsifiers, processing agents and additives and aqueous phase liquid (or oil phase liquid) containing emulsifiers, processing agents and additives are combined and subject to high-speed shearing (or stirring) at the same time. In some embodiments, high-shear mixing is simultaneously applied while mixing oil phase liquid containing emulsifiers, processing agents and additives, and aqueous phase liquid containing emulsifiers. In some embodiments, the isotonic lipid emulsion is formed from a concentrated, hypertonic lipid emulsion by dilution.

In some embodiments, the mixed liquid needs to be dispersed. For example, it can be stirred, sheared, heated, and pressurized. After dispersion, the oil-in-water (O/W) dispersion state is obtained, with the particle size ranging from about 50 nm to about 10 μm. In some embodiments, oil-in-water (O/W) lipid particles having a particle size ranging from about 10 nm to about 1000 nm (e.g., 10 nm to 500 nm, 10 nm to 400 nm, 10 nm to 300 nm, 10 nm to 250 nm, 10 nm to 200 nm, 10 nm to 150 nm, 10 nm to 1000 nm, 10 nm to 80 nm) can be obtained with (or without) high-pressure homogenization. In some embodiments, the isotonic lipid emulsion is an oil-in-water emulsion. In some embodiments, the oil component in the isotonic lipid emulsion has a concentration of from 2 g/100 mL to about 20 g/100 mL.

In some embodiments, the isotonic lipid emulsion is formulated for enteral administration. In some embodiments, the isotonic lipid emulsion is formulated for enteral administration through an oral, nasal, or jejunal feeding tube.

(1) Characteristics of the Isotonic Lipid Emulsion

a. Emulsion Particle Size

In some embodiments, at least about 10%, 20%, 30%, 40%, 50% 60%, 70%, 80% or 90% of the lipid emulsion particles can be about 10 nm to about 900 nm in its largest dimension, e.g., about 10 nm to about 800 nm, about 10 nm to about 700 nm, about 10 nm to about 600 nm, about 10 nm to about 500 nm, about 10 nm to about 400 nm, about 10 nm to about 300 nm, about 10 nm to about 250 nm, about 10 nm to about 230 nm, about 10 nm to about 200 nm, about 10 nm to about 150 nm, about 10 nm to about 120 nm, about 10 nm to about 100 nm, about 10 nm to about 75 nm, or about 10 nm to about 50 nm in its largest dimension. In some embodiments, at least about 10%, 20%, 30%, 40%, 50% 60%, 70%, 80% or 90% of the lipid emulsion particles can be about 25 nm to about 900 nm in its largest dimension, e.g., about 25 nm to about 800 nm, about 25 nm to about 700 nm, about 25 nm to about 600 nm, about 25 nm to about 500 nm, about 25 nm to about 400 nm, about 25 nm to about 300 nm, about 25 nm to about 250 nm, about 25 nm to about 230 nm, about 25 nm to about 200 nm, about 25 nm to about 150 nm, about 25 nm to about 120 nm, about 25 nm to about 100 nm, about 25 nm to about 75 nm, or about 25 nm to about 50 nm in its largest dimension. In some embodiments, at least about 10%, 20%, 30%, 40%, 50% 60%, 70%, 80% or 90% of the lipid emulsion particles can be about 50 nm to about 900 nm in its largest dimension, e.g., about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 250 nm, about 50 nm to about 230 nm, about 50 nm to about 200 nm, about 50 nm to about 150 nm, about 50 nm to about 120 nm, about 50 nm to about 100 nm, or about 50 nm to about 75 nm in its largest dimension.

In some embodiments, the lipid emulsion particles may have a largest dimension (e.g., diameter) of less than about 900 nm. In some embodiments, the lipid emulsion particles have the largest dimension of less than about 800 nm. In some embodiments, the lipid emulsion particles have the largest dimension of less than about 700 nm. In some embodiments, the lipid emulsion particles have the largest dimension of less than about 600 nm. In some embodiments, the lipid emulsion particles have the largest dimension of less than about 500 nm. In some embodiments, the lipid emulsion particles have the largest dimension of less than about 400 nm. In some embodiments, the lipid emulsion particles have the largest dimension of less than about 300 nm. In some embodiments, the lipid emulsion particles have the largest dimension of less than about 250 nm. In some embodiments, the lipid emulsion particles have the largest dimension of less than about 230 nm. In some embodiments, the lipid emulsion particles have the largest dimension of less than about 200 nm. In some embodiments, the lipid emulsion particles have the largest dimension of less than about 180 nm. In some embodiments, the lipid emulsion particles have the largest dimension of less than about 150 nm. In some embodiments, the lipid emulsion particles have the largest dimension of less than about 120 nm. In some embodiments, the lipid emulsion particles have the largest dimension of less than about 100 nm. In some embodiments, the lipid emulsion particles have the largest dimension of less than about 75 nm. In some embodiments, the lipid emulsion particles have the largest dimension of less than about 50 nm.

In some embodiments, the lipid emulsion particles can have an average diameter of about 10 nm to about 900 nm, e.g., about 10 nm to about 800 nm, about 10 nm to about 700 nm, about 10 nm to about 600 nm, about 10 nm to about 500 nm, about 10 nm to about 400 nm, about 10 nm to about 300 nm, about 10 nm to about 250 nm, about 10 nm to about 230 nm, about 10 nm to about 200 nm, about 10 nm to about 150 nm, about 10 nm to about 120 nm, about 10 nm to about 100 nm, about 10 nm to about 75 nm, or about 10 nm to about 50 nm. In some embodiments, the lipid emulsion particles can have an average diameter of about 25 nm to about 900 nm, e.g., about 25 nm to about 800 nm, about 25 nm to about 700 nm, about 25 nm to about 600 nm, about 25 nm to about 500 nm, about 25 nm to about 400 nm, about 25 nm to about 300 nm, about 25 nm to about 250 nm, about 25 nm to about 230 nm, about 25 nm to about 200 nm, about 25 nm to about 150 nm, about 25 nm to about 120 nm, about 25 nm to about 100 nm, about 25 nm to about 75 nm, or about 25 nm to about 50 nm. In some embodiments, the lipid emulsion particles can have an average diameter of about 50 nm to about 900 nm, e.g., about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 250 nm, about 50 nm to about 230 nm, about 50 nm to about 200 nm, about 50 nm to about 150 nm, about 50 nm to about 120 nm, about 50 nm to about 100 nm, or about 50 nm to about 75 nm.

In some embodiments, the average particle size of the emulsion particles is between about 10 nm and about 250 nm. In some embodiments, at least 50% of the emulsion particles have particle sizes of 230 nm or less. In some embodiments, at least 90% of the emulsion particles have particle sizes of 600 nm or less.

It may be desirable to use a population of the lipid emulsion particles that is relatively uniform in terms of size, shape, and/or composition so that each particle of the lipid emulsion particles has similar properties. For example, at least 80%, at least 90%, or at least 95% of the particles may have a diameter or largest dimension that falls within 5%, 10%, or 20% of the average diameter or largest dimension. In some embodiments, a population of the lipid emulsion particles may be heterogeneous with respect to size, shape, and/or composition.

In some embodiments, one or more substantially uniform populations of the lipid emulsion particles are used, e.g., 2, 3, 4, 5, or more substantially uniform populations having distinguishable properties (e.g., size, optical property) or associated with different therapeutic agents. In some embodiments, the disclosed composition or pharmaceutical composition may include two or more populations of the lipid emulsion particles. It will be appreciated that a combination of two or more populations having distinguishable properties can be considered to be a single population.

b. pH Values

In some embodiments, the pH of the lipid emulsion of the present invention may need to be adjusted to a physiologically acceptable range between about 3.0 and about 8.0. Depending on the sterilization process, the following two types are included: (1) when the F0 value of sterilization temperature and time is less than or equal to 1, the preferred pH is between about 3.0 and about 4.5; and (2) when the F0 value of sterilization temperature and time is greater than or equal to 8, the preferred pH is between about 6.5 and about 7.5. “F0” is defined as the number of equivalent minutes of steam sterilization at temperature 121.1° C. (250° F.) delivered to a container or unit of product calculated using a z-value of 10° C. Processing agents and additives can be added to the mixture of medium-chain fatty acid and Omega-3 fatty acid prior to emulsification or can be added to the emulsion prior to sterilization.

In some embodiments, the pH regulator may be used to adjust the pH of the lipid emulsion. The pH regulator may be acidic or basic compounds such as acetic acid, adipic acid, ammonium aluminum sulfate, ammonium, bicarbonate, ammonium carbonate, ammonium citrate, dibasic, ammonium citrate, monobasic, ammonium hydroxide, ammonium phosphate, dibasic, ammonium phosphate, monobasic, calcium acetate, calcium acid pyrophosphate, calcium carbonate, calcium chloride, calcium citrate, calcium fumarate, calcium gluconate, calcium hydroxide, calcium lactate, calcium oxide, calcium phosphate, dibasic, calcium phosphate, monobasic, calcium phosphate, tribasic, calcium sulfate, citric acid, fumaric acid, gluconic acid, hydrochloric acid, lactic acid, magnesium carbonate, magnesium citrate, magnesium fumarate, magnesium hydroxide, magnesium oxide, magnesium phosphate, magnesium sulfate, malic acid, phosphoric acid, potassium acid tartrate, potassium aluminum sulfate, potassium bicarbonate, potassium carbonate, potassium chloride, potassium citrate, potassium fumarate, potassium hydroxide, potassium lactate, potassium phosphate, dibasic, potassium phosphate, tribasic, potassium sulfate, sodium acetate, sodium bicarbonate, sodium bisulfate, sodium carbonate, sodium citrate, sodium hydroxide, sodium phosphate, dibasic, sodium phosphate, monobasic, sulphuric acid, tartaric acid, or combination thereof.

In some embodiments, the isotonic lipid emulsion has a pH between about 3.0 and about 8.0. In some embodiments, the isotonic lipid emulsion has a pH between about 3.0 and about 4.5 when the F0 value of sterilization temperature and time is less than or equal to 1. In some embodiments, the isotonic lipid emulsion has a pH between about 6.5 and about 7.5 when the F0 value of sterilization temperature and time is greater than or equal to 8.

c. Osmotic Pressure

The lipid emulsion of the present invention is an isotonic liquid having an osmotic pressure from 280 mmol/L to 320 mmol/L. For stability and isotonicity, approximately 2% to 5% weight (based on the total weight of the emulsion) of stabilizer or isotonic additive, such as polyhydroxy alcohol, may be included. Glycerol, sorbitol, xylitol, or glucose are preferred. In some embodiments, the osmotic pressure of the isotonic liquid may be from about 280 mmol/L to about 320 mmol/L, which is equivalent to the osmotic pressure of the human body that is also from 280 mmol/L to 320 mmol/L.

Unexpectedly, osmotic pressure plays an important role in absorption of lipid particles. For example, generally, the absorption rate of unisotonic or hypertonic lipid particles by the small intestine is lower than that of the isotonic lipid particles. This is true even when the unisotonic or hypertonic lipid particles (e.g., 50 nm to 100 nm) have smaller particle sizes than the isotonic lipid particles (e.g., 100 nm to 500 nm).

d. Lipid Components

The lipid emulsion of the present invention containing Omega-3 fatty acid and MCT fatty acid is based on the total amount of lipid in the emulsion and comprises: (i) about 1% to about 78% by weight of MCT, and (ii) about 22% to about 99% by weight of Omega-3 fatty acids.

In some embodiments, the lipid emulsion comprises, for example, about 1% to about 75%, about 1% to about 72%, about 1% to about 70%, about 1% to about 68%, about 1% to about 66%, about 1% to about 64%, about 1% to about 62%, about 1% to about 60%, about 1% to about 58%, about 1% to about 56%, about 1% to about 54%, about 1% to about 52%, about 1% to about 50%, about 1% to about 48%, about 1% to about 46%, about 1% to about 44%, about 1% to about 42%, about 1% to about 40%, about 1% to about 38%, about 1% to about 36%, about 1% to about 34%, about 1% to about 32%, about 1% to about 30%, by weight of MCT.

In some embodiments, the lipid emulsion comprises, for example, about 24% to about 99%, about 26% to about 99%, about 28% to about 99%, about 30% to about 99%, about 32% to about 99%, about 34% to about 99%, about 36% to about 99%, about 38% to about 99%, about 40% to about 99%, about 42% to about 99%, about 44% to about 99%, about 46% to about 99%, about 48% to about 99%, about 50% to about 99%, about 52% to about 99%, about 54% to about 99%, about 56% to about 99%, about 58% to about 99%, about 60% to about 99%, about 62% to about 99%, about 64% to about 99%, about 66% to about 99%, about 68% to about 99%, about 70% to about 99%, by weight of Omega-3 fatty acids.

Surprisingly, as demonstrated in this disclosure, by using the lipid emulsion of the present invention, the Omega-3 fatty acid content on the cell membrane of some key organs and tissues was significantly increased. The lipid emulsion of the present invention is more effective than similar emulsion or oils and plant oil agent comprising fish oil as the sole source of triglycerides. It was further demonstrated that the ratio of MCT fatty acid to Omega-3 fatty acid has a significant effect on the results obtained.

The MCT fatty acid described therein contains fatty acid having 6 to 14 carbon atoms. In some embodiments, the MCT contains at least 90% weight of caprylic acid (C8) and/or capric acid (C10). In some embodiments, the MCT in the lipid emulsion of the present invention is about 10% to about 35% of the total lipid content of the lipid emulsion. The source of the MCT may include, without limitation, plant extract, animal extract, and synthetic fatty acid.

The Omega-3 fatty acids described include eicosapentaenoic acid (EPA, 20:5 ω-3) and docosahexaenoic acid (DHA, 22:6 ω-3), which are mostly polyunsaturated fatty acids that can not be synthesized by human body and thus are essential and biologically important. The Omega-3 fatty acid described may be derived from fish oil that is about 22% to about 99% of the total lipid content of the lipid emulsion, e.g., about 65% to about 90% of the total lipid content of the lipid emulsion. The fish oil described is selected from natural fish oil, processed fish oil, purified fish oil concentrate, (re)esterified synthetic fish oil and mixtures thereof, and fish oil extracted using bioengineering from microorganisms such as algae or selected from krill oil. When the fatty acid methyl ester of fish oil concentrate is used, the triglyceride of the fish oil described contains about 25% to about 95% EPA and about 12% to about 95% DHA.

The total lipid content of the isotonic lipid emulsion of the present invention, based on the weight of the lipid emulsion, is 5% to 60% by weight, e.g., 5% to 50%, 5% to 45%, 5% to 40%, 5% to 35%, 5% to 30%, 5% to 25%, 5% to 20%, 5% to 15%, 5% to 10%, 15% to 50%, 10% to 45%, 10% to 40%, 10% to 35%, 10% to 30%, 10% to 25%, 10% to 20%, 10% to 15%, 15% to 50%, 15% to 45%, 15% to 40%, 15% to 35%, 15% to 30%, 15% to 25%, or 15% to 20% by weight.

The lipid emulsion of the present invention covers the distinctive fishy smell of the lipid by taste modifying and taste-masking techniques, thus making the taste of the lipid emulsion pleasant. The lipid emulsion is of oil-in-water (O/W) type, in which water molecules encapsulate lipid, reducing contact between the lipid and the taste bud. In addition, flavoring agents, such as edible essence, and sweetener can be added to mask the fishy smell of the lipid. Also, a small amount of antioxidants can be added to further reduce the oxidation of unsaturated fatty acids in order to avoid taste changes during long-term placement and improve administration experience.

The lipid emulsion of the present invention is a fatty acid composition of Omega-3 fatty acid, suitable for enteral administration. It can be rapidly absorbed by the cells and efficiently enriched on cell membranes. The lipid emulsion is capable of rapidly modifying the fatty acid composition of organ and tissue cell membranes.

Presently, absorption of fatty acids by the human body was determined mainly based on detecting fatty acids content in plasma. However, this fatty acid content in plasma cannot reflect the degree of Omega-3 fatty acid enrichment on the cell membrane of organs and tissues. Instead, the testing of fatty acids on the blood cell membrane can more accurately reflect the degree of enrichment of fatty acids on the cell membrane of organs and tissues. Therefore, testing the fatty acid content on the cell membrane is more valuable than testing the fatty acid content in plasma.

As an unexpected discovery, this disclosure demonstrated that using the lipid emulsion of the present invention in 43 cases of human experiments up to 4 weeks, on day 7 after oral administration of the emulsion, EPA in leukocyte membrane phospholipids increased by 165% and DHA increased by 34% compared to day 0, EPA in platelet cell membrane phospholipids increased by 278%, and DHA increased by 25%. On day 28 after oral administration of the emulsion, EPA in leukocyte membrane phospholipids increased by 260%, DHA increased by 67% compared to day 0, EPA in platelet cell membrane phospholipids increased by 409%, and DHA increased by 47%. As can be seen from the experimental results, the lipid emulsion as disclosed here is capable of rapidly and efficiently enriching Omega-3 fatty acids onto the cell membrane of organs and tissues, rapidly changing the composition of fatty acids on the membranes of these organs and tissues.

The lipid emulsion of the present invention comprising Omega-3 fatty acid and MCT fatty acid is suitable for rapidly improving the metabolic syndrome of people deficient in Omega-3 fatty acids. Long-term deficiency of Omega-3 fatty acids can lead to a series of metabolic syndromes, such as insulin signal and glucose homeostasis deficiency; increased blood pressure and cardiomegaly; increased fat (triglyceride) concentrations in plasma, liver, and muscle; deteriorating inflammation and oxidation; reduced platelet activation and increased risk of thrombosis; tissue perfusion changes, etc. Studies have shown that as long as a sufficient amount of Omega-3 fatty acids are supplemented, these metabolic syndromes caused by a lack of Omega-3 fatty acids will rapidly improve until returning to normal.

(2) Other Ingredients

In some embodiments, the isotonic lipid emulsion further comprises an additional agent such as emulsifier, emulsifying aid, stabilizer, antioxidant, ion antagonism, defoaming agent, natural (or synthetic) flavoring, natural (or synthetic) fragrance, and agents for balancing osmolarity.

a. Emulsifier

Oil-in-water emulsions are capable of drastically increasing the bioavailability of lipophilic material. Additionally, emulsions made using emulsifiers and/or surfactants offer a method of encapsulation for active components that protects them from environmental stresses that may cause unwanted degradation. Depending on the ingredients and processing conditions, emulsions can be created with specific particle sizes ranging from several microns down to nanometers with both high and low polydispersity. In this way, the lipid emulsion may be tailor-made for specific applications. For instance, to reduce onset times, fine emulsion particles can be made with high surface areas that facilitate rapid uptake in the body.

Emulsifiers and surfactants can be used for encapsulating the oily fraction and partitioning it from the aqueous phase. Depending on the type of emulsifier/surfactant used, emulsions can be made from micelles or liposomes. Micelles have the oily fraction contained in the core, encapsulated in emulsifier/surfactant that partitions the oil phase from the aqueous phase. Liposomes are bilayer systems where an aqueous core is surrounded by a bilayer system containing the oil phase inside the bilayer.

The type of emulsifier/surfactant chosen has an impact on the particle size, emulsion stability, active ingredient release profile, and flavor profile. Additionally, the amount of emulsifier used has an impact on the particle size, the release profile, and the encapsulation efficiency. Semi-natural and synthetic emulsifiers/surfactants can be used to create the emulsion systems described herein. Such emulsions can have sufficiently small particle sizes that impart intrinsic emulsion stability and high dispersibility with a high surface area that facilitates rapid uptake in the body.

The emulsifier can be a physiologically acceptable emulsifier (or surfactant), which may be one or more selected from soybean phospholipids, sucrose esters, citric acid fatty acid glycerides, fatty acid glycerolipids, polysorbates, fatty acid sorbitans, cyclodextrins, polyoxyethylene fatty acid esters, polyoxyethylene polyoxypropylene copolymers, polyoxyethylene fatty alcohol ethers, polyethylene glycol, poloxamer, chitin, chitosan, cholic acid, and its salts. In some embodiments, the emulsifier can be soybean phospholipids, fatty acid glycerides, or polyoxyethylene fatty acid esters.

b. Antioxidants/Emulsification Aids

The processing agents described therein may include antioxidants, emulsification aids, etc. Antioxidants can be used to prevent or at least inhibit or mitigate the degradation of cannabinoids from oxidation. The antioxidant may be one or more selected from sodium sulfite, sodium hydrogen sulfite, sodium pyrosulfate, vitamin C esters thereof, and tocopherols and esters thereof, preferably vitamin C and mixed tocopherol; the emulsification aid may be selected from alkali metal salts with long-chain C16 to C20 fatty acids, preferably sodium salt thereof.

In some embodiments, antioxidants can be any one of: ethanol, polyethylene glycol 300, polyethylene glycol 400, propylene glycol, propylene carbonate, N-methyl-2-pyrrolidones, dimethylacetamide, dimethyl sulfoxide, hydroxypropyl-P-cyclodextrins, sulfobutylether-p-cyclodextrin, a-cyclodextrin, HSPC phospholipid, DSPG phospholipid, DMPC phospholipid, DMPG phospholipid, ascorbyl palmitate, butylated hydroxyanisole, butylated hydroxyanisole, propyl gallate, a-tocopherol, γ-tocopherol, propyl gallate, lecithin, Vitamin E tocopherol, sesamin, sesamol, sesamolin, alpha-tocopherol, ascorbic acid, ascorbylpalmitate, fumaric acid, malic acid, sodium metabisulfite, and EDTA. Specific antioxidant examples include, but are not limited to: Ascorbic Acid: 0.001 to 5% w/w of emulsion system, Vitamin E Tocopherol: 0.001 to 5% w/w of emulsion system, Tocopherol: 0.001 to 5% w/w of emulsion system, and combinations of ascorbic acid, vitamin E tocopherol, and tocopherol can be used for this invention.

c. Preservatives

The lipid emulsion disclosed herein may further include a preservative. Oil-in-water emulsions are aqueous in nature and susceptible to microbial growth. Preservatives can be used to prevent microbial spoilage. These preservatives include: methylparabens, ethylparabens, propylparabens, butylparabens, sorbic acid, acetic acid, propionic acid, sulfites, nitrites, sodium sorbate, potassium sorbate, calcium sorbate, benzoic acid, sodium benzonate, potassium benzonate, calcium benzoate, sodium metabisulfite, propylene glycol, benzaldehyde, butylated hydroxytoluene, butylated hydroxyanisole, formaldehyde donors, essential oils, citric acid, monoglyceride, phenol, mercury components and any combination thereof.

Amongst useful preservatives include chelating agents some of which are listed above and other chelating agents, e.g., nitrilotriacetic acid (NTA); ethylenediaminetetracetic acid (EDTA), hydroxyethylethylenediaminetriacetic acid (HEDTA), diethylenetriaminepentaacetic acid (DPTA), 1,2-Diaminopropanetetraacetic acid (1,2-PDTA); 1,3-Diaminopropanetetraacetic acid (1,3-PDTA); 2,2-ethylenedioxybis[ethyliminodi(acetic acid)](EGTA); 1,10-bis(2-pyridylmethyl)-1,4,7,10-tetradecane (BPTETA); ethylenediamine (EDAMINE); Trans-1,2-diaminocyclohexane-N,N,N′,N′-tetraacetic acid (CDTA); ethylenediamine-N,N′-diacetate (EDDA); phenazine methosulphate (PMS); 2,6-Dichloro-indophenol (DCPIP); Bis(carboxymethyl)diaza-18-crown-6 (CROWN); porphine; chlorophyll; dimercaprol (2,3-Dimercapto-1-propanol); citric acid; tartaric acid; fumaric acid; malic acid; and salts thereof. The preservatives listed above are exemplary, but each preservative must be evaluated in each formulation, to assure the compatibility and efficacy of the preservative. Methods for evaluating the efficacy of preservatives in pharmaceutical formulations are known to those skilled in the art.

Additionally, the pH of the emulsion can be lowered to prevent or retard microbial growth. Lowering the pH below 4.0 is sufficiently low enough to prevent microbial growth for a minimum of 1 month.

d. Sweetener

In the instance where auxiliary sweeteners are utilized, the formulation may include those sweeteners well known in the art, including both natural and artificial sweeteners. Thus, additional sweeteners may be chosen from the following non-limiting list: water-soluble sweetening agents such as monosaccharides, disaccharides, and polysaccharides such as xylose, ribose, glucose, mannose, galactose, fructose, high fructose corn syrup, dextrose, sucrose, sugar, maltose, partially hydrolyzed starch, or corn syrup solids and sugar alcohols such as sorbitol, xylitol, mannitol, and mixtures thereof.

In general, the amount of sweetener will vary with the desired amount of sweeteners selected for a particular formulation. This amount will typically be 0.001% to about 90% by weight, per volume of the final composition, when using an easily extractable sweetener. The water-soluble sweeteners described above are preferably used in amounts of about 5% to about 70% by weight per volume, and most preferably from about 10% to about 50% by weight per volume of the final liquid composition. In contrast, the artificial sweeteners (e.g., sucralose, acesulfame K, and dipeptide based sweeteners) are used in amounts of about 0.005% to about 5.0% and most preferably about 0.01% to about 2.5% by weight per volume of the final liquid composition. These amounts are ordinarily necessary to achieve a desired level of sweetness independent from the flavor level achieved from flavor oils.

e. Flavoring Agents

Suitable flavorings include both natural and artificial flavors, and mints such as peppermint, menthol, artificial vanilla, cinnamon, various fruit flavors, both individual and mixed, essential oils (i.e., thymol, eucalyptol, menthol, and methyl salicylate) and the like are contemplated. The amount of flavoring employed is normally a matter of preference subject to such factors as flavor type, individual flavor, and strength desired. Thus, the amount may be varied in order to obtain the result desired in the final product. Such variations are within the capabilities of those skilled in the art without the need for undue experimentation. The flavorings are generally utilized in amounts that will vary depending upon the individual flavor, and may, for example, range in amounts of about 0.01 to about 3% by weight per volume of the final composition weight.

f. Colorants

The colorants useful in the present invention, include the pigments such as titanium dioxide that may be incorporated in amounts of up to about 1% by weight per volume, e.g., up to about 0.6% by weight per volume. Also, the colorants may include dyes suitable for food, drug, and cosmetic applications, and known as D&C and F.D. & C. dyes and the like. The materials acceptable for the foregoing spectrum of use are preferably water-soluble. Illustrative examples include indigoid dye, known as F.D. & C. Blue No. 2, which is the disodium salt of 5,5′indigotindisulfonic acid. Similarly, the dye known as F.D. & C. Green No. 1 comprises a triphenylmethane dye and is the monosodium salt of 4-[4-N-ethyl p-sulfobenzylamino)diphenylmethylene]-[1-(N-ethyl-N-p-sulfoniumbenzyl)-2,5-cyclohexadienimine]. A full recitation of all F.D. & C. and D. & C. and their corresponding chemical structures may be found in the Kirk-Othmer Encyclopedia of Chemical Technology, in Volume 5, at Pages 857-884, which text is accordingly incorporated herein by reference.

(3) Compositions

In another aspect, this disclosure also provides a composition comprising the isotonic lipid emulsion, as described above. Also within the scope of this disclosure is a pharmaceutical composition comprising the isotonic lipid emulsion described above and a pharmaceutically acceptable carrier.

As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use.

The term “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of one or more components of the invention, and are physiologically acceptable to the subject. Supplementary active compounds may also be incorporated into the compositions.

In some embodiments, the composition or the pharmaceutical composition may include a therapeutic agent, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

Also within the scope of this disclosure is a food or drink additive comprising the isotonic lipid emulsion as described above. In some embodiments, the food or drink additive is formulated for food or drink selected from the group consisting of water, fruit juice, and vegetable juice, or for natural (or synthetic) flavoring and fragrance to prepare one or more foods.

Also within the scope of this disclosure is a product containing the isotonic lipid emulsion. In some embodiments, the product can be in packs in a form ready for administration, e.g., a blister pack, a bottle, syringes, foil packs, pouches, or other suitable containers. In some embodiments, the compositions are in concentrated form in packs, optionally with a diluent.

The composition can be administered to any patient in need thereof. Although preferred patients are human, animals, especially domestic animals such as dogs, cats, horses, cattle, sheep, goats, and fowl, may also be treated with the composition. The amount of the active ingredients to be administered is chosen based on the amount which provides the desired dose to the patient in need of such treatment to alleviate symptoms or treat a condition.

(4) Kits

A composition described herein can be provided in a kit. In one embodiment, the kit includes (a) a container that contains the composition, and optionally (b) informational material. The informational material can be descriptive, instructional, marketing or other material that relates to the methods described herein and/or the use of the agents for therapeutic benefit. In an embodiment, the kit also includes an additional therapeutic agent, as described above. For example, the kit includes a first container that contains the composition and a second container for the additional therapeutic agent.

The informational material of the kits is not limited in its form. In one embodiment, the informational material can include information about production of the composition, concentration, date of expiration, batch or production site information, and so forth. In one embodiment, the informational material relates to methods of administering the composition, e.g., in a suitable dose, dosage form, or mode of administration (e.g., a dose, dosage form, or mode of administration described herein), to treat a subject in need thereof. In one embodiment, the instructions provide a dosing regimen, dosing schedule, and/or route of administration of the composition or the additional therapeutic agent. The information can be provided in a variety of formats, including printed text, computer-readable material, video recording, or audio recording, or information that contains a link or address to substantive material.

The kit can include one or more containers for the composition. In some embodiments, the kit contains separate containers, dividers or compartments for the composition and informational material. For example, the composition can be contained in a bottle or vial, and the informational material can be contained in a plastic sleeve or packet. In other embodiments, the separate elements of the kit are contained within a single, undivided container. For example, the composition is contained in a bottle or vial that has attached thereto the informational material in the form of a label. In some embodiments, the kit includes a plurality (e.g., a pack) of individual containers, each containing one or more unit dosage forms (e.g., a dosage form described herein) of the agents.

The kit optionally includes a device suitable for administration of the composition or other suitable delivery device. The device can be provided pre-loaded with one or both of the agents or can be empty, but suitable for loading.

### B. METHODS OF USE OF THE LIPID EMULSION

In another aspect, this disclosure further provides a method of administering a lipid emulsion. The method comprises administering enterally a dose of the isotonic lipid emulsion, the composition, or the pharmaceutical composition, as described above, to a subject in need thereof. In some embodiments, the subject is a mammal, such as human.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

In yet another aspect, this disclosure additionally provides a method for treating a human having Omega-3 fatty acid deficiency. The method comprises enterally administering to the human an effective dosage amount of the isotonic lipid emulsion, the composition, or the pharmaceutical composition, as described above, thereby increasing enrichment of Omega-3 fatty acids on the cell membranes of human tissues and organs by at least 10% compared to a predetermined reference value.

In some embodiments, the organs are selected from the group consisting of heart, kidney, brain, liver, lung, and adipose tissue. In some embodiments, the tissues are selected from the group consisting of endothelium, white blood cell, platelet, and immune cell.

In some embodiments, the predetermined value is obtained from the subject prior to the administration of the isotonic lipid emulsion, the composition, or the pharmaceutical composition, as described above. In some embodiments, the predetermined value is obtained from a control subject or a group of individuals who do not have Omega-3 fatty acid deficiency or have not been diagnosed with Omega-3 fatty acid deficiency. In some embodiments, the predetermined value is obtained based on average levels of Omega-3 fatty acid in a control population, for example, a population that does have Omega-3 fatty acid deficiency. In some embodiments, the predetermined value is obtained based on a median or median level of a set of individuals in which patients with Omega-3 fatty acid deficiency are included.

In some embodiments, the predetermined reference value is a level of EPA on day 0, such as EPA in leukocyte membrane phospholipids or EPA in platelet cell membrane phospholipids. In some embodiments, the predetermined reference value is a level of DHA on day 0, such as DHA in leukocyte membrane phospholipids or DHA in platelet cell membrane phospholipids. As demonstrated in this disclosure, the lipid emulsion of the present invention is capable of rapidly and efficiently enriching Omega-3 fatty acids onto the cell membrane of organs and tissues, rapidly changing the composition of fatty acids on the membranes of these organs and tissues. For example, using the lipid emulsion of the present invention in 43 cases of human experiments up to 4 weeks, on day 7 after oral administration of the emulsion, EPA in leukocyte membrane phospholipids increased by 165% and DHA increased by 34% compared to day 0. EPA in platelet cell membrane phospholipids increased by 278%, and DHA increased by 25%. On day 28 after oral administration of the emulsion, EPA in leukocyte membrane phospholipids increased by 260% and DHA increased by 67% compared to day 0; EPA in platelet cell membrane phospholipids increased by 409%, and DHA increased by 47%.

In some embodiments, the human has a condition selected from the group consisting of a systemic inflammatory response syndrome, a respiratory distress syndrome, a nutritional and/or dietary cause of liver disease, an iatrogenic cause of liver disease, a pathological cause of liver disease, an immune modulation, head trauma, postoperative surgical stress, a myocardial infarction, cystic fibrosis and a combination thereof.

In some embodiments, the human is in need of rapidly supplementing Omega-3 fatty acids to improve metabolic syndrome, or to benefit from the efficacy of Omega-3 fatty acids in modulating inflammation, prevention of premature birth, myocardial ischemia or infarction, transient local cerebral ischemia or stroke, autoimmunity, and thrombotic diseases, organ transplantation, acute phase response, acute respiratory distress syndrome, inflammatory bowel syndrome, and hypertriglyceridemia.

In some embodiments, the lipid emulsion is enterally administered. In some embodiments, the enteral administration is carried out through an oral, nasal, or jejunal feeding tube.

### C. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder, or condition; and generally counteracting a disease, symptom, disorder or pathological condition.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), etc.). Pharmaceutical grade further incorporates suitability for administration by means including enteral, topical, ocular, parenteral, nasal, pulmonary tract, mucosal, vaginal, rectal, intravenous, and the like.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

The term “mixture” as used herein, refers to a combination of elements, that are interspersed and not in any particular order. A mixture is heterogeneous and not spatially separable into its different constituents. Examples of mixtures of elements include a number of different elements that are dissolved in the same aqueous solution or a number of different elements attached to a solid support at random or in no particular order in which the different elements are not spatially distinct. In other words, a mixture is not addressable.

As used herein, “treatment” or “treating,” or “palliating” or “ameliorating” are used interchangeably. These terms refer to an approach for obtaining beneficial or desired results, including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

As used herein, the term “modulate” is meant to refer to any change in biological state, i.e., increasing, decreasing, and the like.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Sample,” “test sample,” and “patient sample” may be used interchangeably herein. The sample can be a sample of serum, urine plasma, amniotic fluid, cerebrospinal fluid, cells, or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. EXAMPLES

The present application will be further described in detail below with reference to specific embodiments and data. These embodiments are only intended to illustrate the present invention, the specific formulation, preparation process, function and effect of the present invention, rather than limiting the scope of the present invention in any means. In the following embodiments, various processes and methods not described in detail are conventional methods well known in the field.

### Example 1

### Comparison of Particle Sizes of Lipid Emulsions Prepared by Different Methods

Different emulsifiers were used, and different preparation methods were set according to the performance of emulsifiers. The particle size distribution of lipid emulsions between different emulsifiers and different methods were compared.

Method: Fish oil and MCT were used as primary raw materials; vitamin C and mixed tocopherol were used as an antioxidant; EDTA calcium disodium was used as an emulsification aid; glycerin was used as isotonic additive; and polyoxyethylene castor oil and soybean phospholipid were selected as emulsifiers.

Two groups were set up according to the emulsifier: Group A (polyoxyethylene castor oil), Group B (soybean phospholipid), and different preparation processes were carried out and described as follows:

TABLE 1
Different emulsifiers and different methods for
preparing emulsions of different particle sizes

| | Group A | Group B |
|---|---|---|
| Step 1 | Prepare aqueous | Prepare aqueous phase, add |
| | phase, add EDTA, | emulsifier, vitamin C, EDTA, |
| | vitamin C | glycerin |
| Step 2 | Add emulsifier, mixed | Prepare oil phase, add MCT, |
| | tocopherol into the | Omega-3 fatty acid, mixed |
| | aqueous phase | tocopherol |
| Step 3 | Add oil phase, MCT, | Prepare disperse phase, mix the |
| | Omega-3 fatty acid | aqueous phase and the oil phase in |
| | | proportion, and pass shearing machine |
| Step 4 | Filtering | Prepare homogeneous phase, the |
| | | dispersed liquid pass homogenizer |
| Step 5 | Filling | Filling |

The particle sizes of the two groups were measured separately after sterilization. The results are summarized in Table 2.

TABLE 2
Particle size distribution of Group A and Group B

| | Group A | Group B |
|---|---|---|
| Average particle size (d · nm) | 58.23 | 134.50 |

Different emulsifiers can all be prepared into lipid emulsions with an average particle size of less than 500 nm, but the particle size of Group A is significantly lower than that of Group B. From appearance, Group A is a transparent, opalescent clear liquid, which is a microemulsion, while Group B is a white uniform milky liquid, which is a lipid emulsion.

### Methods for Particle Size Characterization

A. Instrument: Malvern Nano-ZS90 (Molecular/Particle Size and Zeta Potential Analyzer) Instrument Setup Parameters:

| Dispersant | Water | |
|---|---|---|
| Dispersant Refractive Index (RI) | 1.330 | |
| Material Refractive Index (RI) | 1.59 | |
| Attenuation (%) | 5%-10% | |
| Viscosity (cP) | 0.8872 | cP |
| Material Absorption | 0.010 | |
| Temperature | 25° | C. |
| Duration | 60 | s |
| Measurement position (mm) | 4.65 | mm |

| | |
|---|---|
| Sample Cell | Plastic cell |

B. Sample Preparation

The following steps were carried out to obtain the samples used in this study.

Step 1: Obtain liquid emulsion in a container (e.g., bag, bottle) and mix well by shaking the container.

Step 2: Transfer 1 mL of liquid emulsion to a 10-mL measuring cup and add the distilled water to the 10-mL mark to dilute the liquid emulation. Mix the sample well. The resulting sample can be slightly turbid.

C. Sample Analysis

Add the above sample from Step 2 to the sample cell to the amount as indicated on the sample cell. Place the sample cell in the Malvern Nano-ZS90 analyzer and carry out the measurement according to the instrument manual.

D. Results

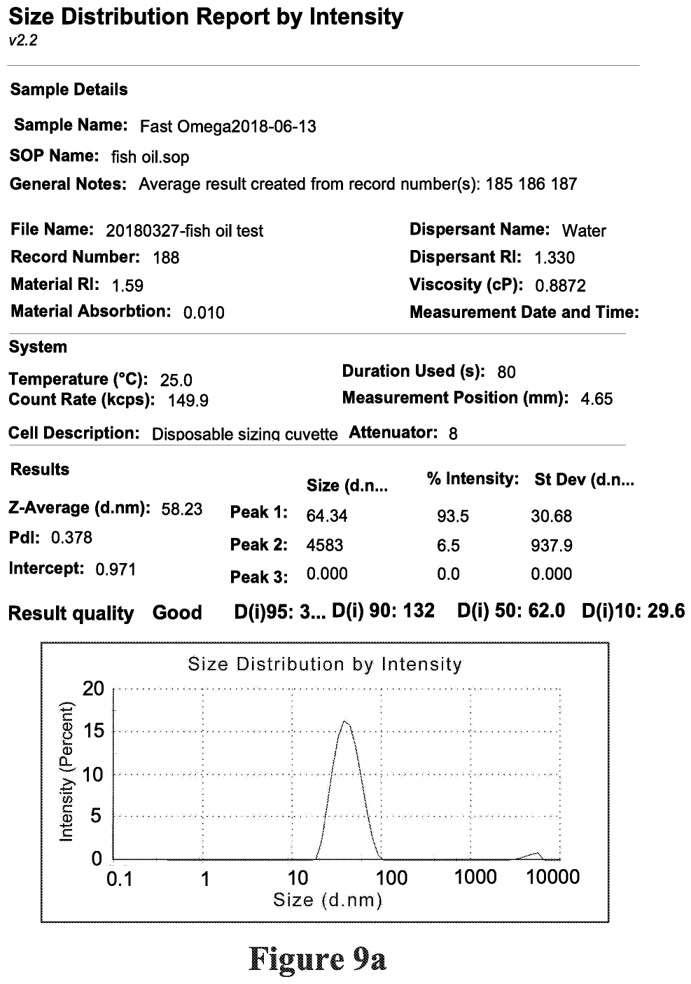

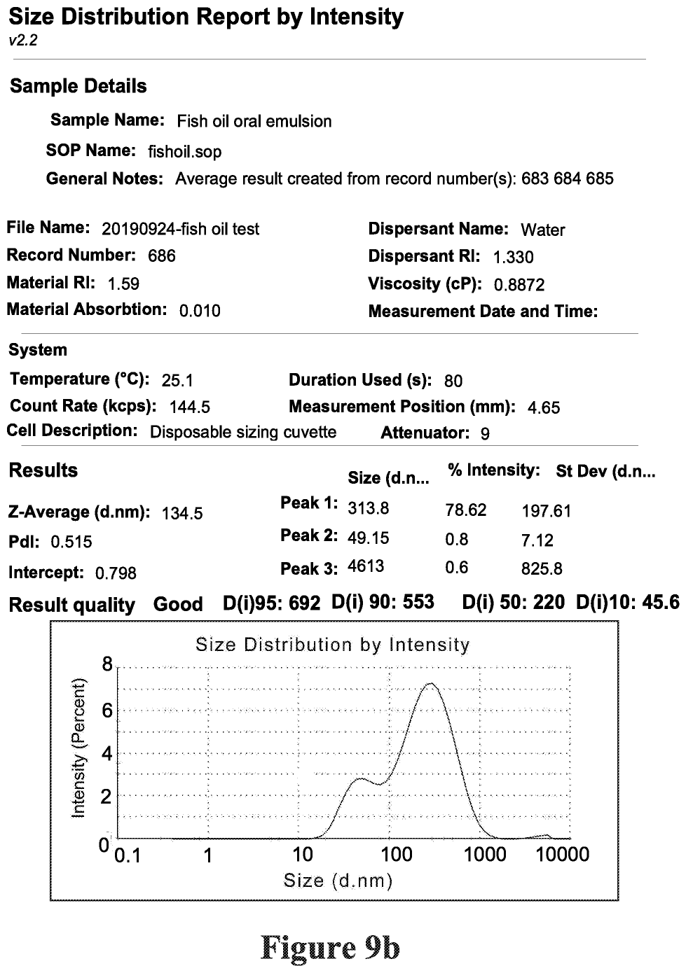

The size distribution reports (e.g., percent intensity vs. size) were generated to provide information such as average size, D95, D90, D50, etc. (FIG. 9a and FIG. 9b).

### Example 2

### Effects of Different pH Values, Different Sterilization Temperatures, and Sterilization Time on the Particle Size of the Lipid Emulsion

The effects of different pH values, different sterilization temperatures, and sterilization time on the particle size of the two groups of emulsion in EXAMPLE 1 were examined. The particle size of the emulsion directly affected the absorption of fatty acids by the small intestine as well as bioavailability, thus having an impact on the effect of the product. Therefore, in consideration of product safety, the stability of the product must also be considered to achieve a reasonable balance.

Method: according to US Federal Regulation 21CFR114, acidified food has a finished equilibrium pH of 4.6 or below, and its sterilization requirements can be carried out according to the requirements of food processing certification bodies from different states. For example, if the pH is above 4.6, the sterilization requires an F0 value>8. In addition, flavoring agent may directly affect the particle size and pH of the product. Therefore, after adding flavoring agent to the liquids of Groups A and B, the inventors examined each group under the same conditions and set the two groups to a pH of 3.5 and a pH of 7.0, respectively. The corresponding sterilization temperature and time were 100° C., 30 minutes and 121° C., 15 minutes. Changes in particle size were investigated, and the results are summarized in Table 3.

TABLE 3
Effects of different pH values, different sterilization temperatures,
and sterilization time on the particle size of the lipid emulsion

| | Group A | Group B |
|---|---|---|
| | Average particle | Average particle |
| | size (d · nm) | size (d · nm) |
| pH value 3.5 | 129.9 | 875.6 |
| Sterilization temperature: 100° C. | | |
| Sterilization time: 30 min | | |
| pH value 7.0 | >10000 | 229.1 |
| Sterilization temperature: 116° C. | | |
| Sterilization time: 30 min | | |

The results indicate that flavoring agent is very sensitive to high temperature, and the flavoring agent used in Group A has a significant impact on its particle size, especially at high temperatures. Thus Group A may not be a good formula. Group B is significantly affected by pH value, but not by the flavoring agent. Thus, Group B may be a good formula. Therefore, for the emulsion formula of Group A, the preferred pH is approximately 3.0 to 4.5, and the F0 value of sterilization temperature and time is less than or equal to 1. For the emulsion formula of Group B, the preferred pH is approximately 6.5 to 7.5, and the F0 value of sterilization temperature and time is greater than or equal to 8. The above characteristics of the lipid emulsion comply with the provisions of the US Federal Regulation 21CFR114.

### Example 3

### Effects of Osmotic Pressure on Intestinal Absorption

Method: the osmotic pressures of Group A and Group B in EXAMPLE 1 were measured respectively. The experiments were carried out in zebrafish. The zebrafish's intestinal system is very similar to the human body, and it is completely transparent, which is very suitable for observing the activity of food or medicine in the intestine. The experimental steps are as follows:

Step 1: Prepare matrix feed, fill them with Group A liquid, Group B liquid, and oil, respectively.

Step 2: Divide the zebrafish into 3 groups of 20 fish each, mark them as Group A, Group B, and Oil Group, and feed them with the corresponding matrix feed for 20 days;

Step 3: On day 0, day 10, and day 20, freeze 5 zebrafish from each group for 48 hours, mince, and homogenize 16 mg/fish to separate the lipid. Separate phospholipids, free fatty acids, and other lipids for testing.

Step 4: Compare the weight percentages of EPA and DHA in total fatty acids of Group A (microemulsion group), Group B (fat emulsion), and Oil Group on day 0, 10, and 20.

The osmotic pressures of Group A and Group B were first compared and shown in Table 4.

TABLE 4
Comparison of osmotic pressure between Group A and Group B

| | Group A | Group B |
|---|---|---|
| Osmotic Pressure (mOsmol/L) | 796 | 298 |

As shown in Table 4, the osmotic pressure of group A is much higher than that of group B, and the particle size of group A is much smaller than that of group B.

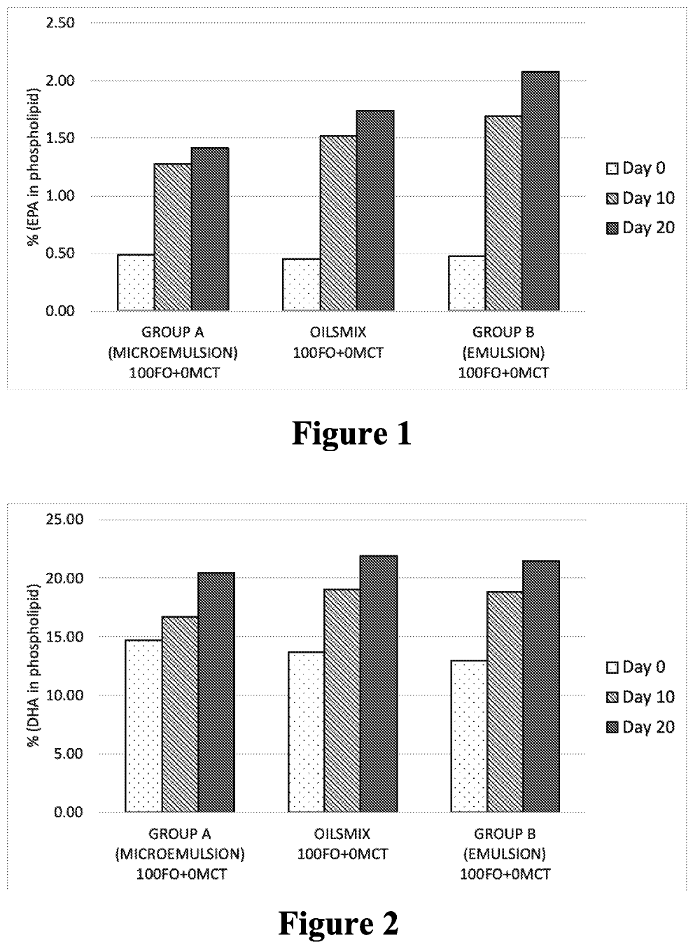

As shown in FIGS. 1 and 2, on day 0, the ratios of EPA/total fatty acid (FF) and DHA/total FF in each group were close, without statistical differences. However, on day 10, both the EPA/FF and DHA/FF of Group A was significantly lower than those of Group B. On day 20, both the EPA/FF and DHA/FF of Group A were significantly lower than those of Group B. The result shows that although the particle size of Group A is small, the high osmotic pressure will affect the absorption of zebrafish intestine, which is enough to offset the advantage of small particle size; though the particle size of Group B is slightly larger, it is still smaller than the particle size of chylomicrons, thus absorbs quickly. Moreover, as Group B is an isotonic liquid, it shows excellent effects without affecting absorption. As a result, Group B absorbs faster and accumulates more quickly on cell membranes of organs and tissues than conventional oil agent.

From the above three examples, it was found that the product obtained by the emulsifier and the preparation process used in Group B has a particle size of less than 500 nm and can be over-sterilized (F0 value>8). In addition, Group B is an isotonic liquid, which has the advantages of fast absorption and higher safety. Therefore, emulsifiers and preparation processes in Group B were used to investigate the effects of different fatty acid ratio compositions on the degree of EPA and DHA enrichment on cell membranes.

### Example 4

### Effects of Different Omega-3 Fatty Acid/MCT Ratios on the Degree of EPA and DHA Enrichment on Cell Membranes

Next, ratios of Omega-3 fatty acids and MCT in lipid emulsions that can enable EPA and DHA to accumulate more quickly on zebrafish organs and tissues were investigated so as to determine a more effective kind of lipid emulsion.

As shown in Table 5, using the same emulsifier and preparation process, 4 groups of lipid emulsions were prepared according to different proportions of Omega-3 fatty acids and MCT-based on the total lipid weight: Group I (100% Omega-3 fatty acids), Group II (90% Omega-3 fatty acids+10% MCT), Group III (70% Omega-3 fatty acids+30% MCT), Group VI (50% Omega-3 fatty acids+50% MCT).

TABLE 5
Lipid emulsion formula of 4 groups

| | Group I | Group II | Group III | Group VI |
|---|---|---|---|---|
| Water (g) | 25.51 | 24.96 | 23.41 | 20.51 |
| Omega-3FA (g) | 5.00 | 5.00 | 5.00 | 5.00 |
| MCT (g) | 0.00 | 0.55 | 2.10 | 5.00 |
| Soybean Phospholipid (g) | 1.549 | 1.549 | 1.549 | 1.549 |
| Glycerin (g) | 0.839 | 0.839 | 0.839 | 0.839 |
| Mixed Tocopherol (g) | 0.013 | 0.013 | 0.013 | 0.013 |
| EDTA (g) | 0.010 | 0.010 | 0.010 | 0.010 |
| Vitamin C (g) | 0.010 | 0.010 | 0.010 | 0.010 |

Preparation Process:

Fish oil and MCT were used as main raw materials; vitamin C and mixed tocopherol were used as an antioxidant; EDTA calcium disodium was used as an emulsification aid; glycerin was used as isotonic additive; and soybean phospholipid was selected as an emulsifier.

Step 1. Prepare aqueous phase, add emulsifier, vitamin C, EDTA, glycerin;

Step 2. Prepare oil phase, add MCT, Omega-3 fatty acid, mixed tocopherol;

Step 3. Prepare the disperse phase, mix the aqueous phase and the oil phase in proportion and pass the shearing machine;

Step 4. Prepare the homogeneous phase, the dispersed liquid pass homogenizer;

Step 5. Filling.

Zebrafish Experiment:

Step 1. Prepare matrix feed, fill the zebrafish with Group I liquid, Group II liquid, Group III liquid and Group VI liquid;

Step 2: Divide the zebrafish into 4 groups of 20 fish each, mark them as Group I, Group II, Group III, and Group VI, and feed them with the corresponding matrix feed for 20 days;

Step 3: On day 0, day 10, and day 20, freeze 5 zebrafish from each group for 48 hours, mince and homogenize 16 mg/fish to separate the lipid. Separate phospholipids, free fatty acids, and other lipids for testing.

Step 4: Compare the weight percentages of EPA and DHA in total fatty acids of Group I, Group II, Group III, and Group VI on day 0, 10, and 20.

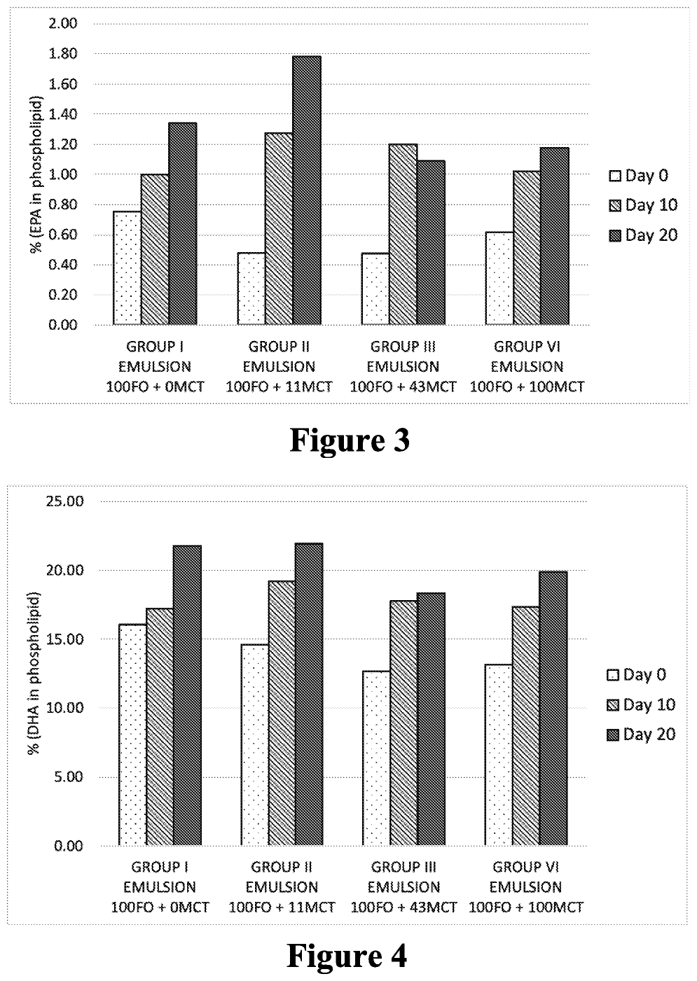

As shown in FIG. 3, the degree of EPA enrichment is higher in Groups II and III, than in Group I and VI on Days 10. Similarly, as shown in FIG. 4, the degree of DHA enrichment is higher in Groups II and III than in Group I and VI on Days 10. It can be seen that by including an appropriate amount of MCT content, the enrichment of EPA and DHA in organs and tissues will be higher and faster, and the optimal ratio of Omega-3 fatty acids/MCT should be between 9/1 to 7/3, that is, based on the total amount of lipids in the emulsion, the optimal combination in the composition of the present invention should be: (i) 10% to 30% weight of MCT, and (ii) 70% to 90% weight of Omega-3 fatty acids.

### Example 5

### The Percentage of Total Lipid Content of MCT+Omega-3 Fatty Acid Based on the Weight of the Lipid Emulsion

To investigate the effective 4 groups in EXAMPLE 4 and calculate the percentage (%) of the weight of MCT+Omega-3 fatty acid to the total lipid content based on the weight of the lipid emulsion.

According to the four groups in Table 5, the weight percentage=[total amount of MCT and Omega-3FA/the total weight of the lipid emulsion]×100%; the mass percentages of Group I, Group II, Group III, and Group VI were calculated respectively.

The weight percentage of Group I is 15%, the weight percentage of Group II is 17%, the weight percentage of Group III is 21%, and the weight percentage of Group VI is 30%. Therefore, based on the weight of the lipid emulsion, the preferred percentage of MCT+Omega-3 fatty acid total lipid content preferably ranges between 10% and 40%.

### Example 6

### Evaluation of Taste-Masking Effect

In this example, the taste-masking effect of different groups in EXAMPLE 4 was investigated. Thirty volunteers (14 males and 16 females) were screened. The volunteers were first screened for fishy taste sensitivity. Pure fish oil was selected as a reference sample and was divided into 5 grades by dilution, a certain I value range was given to each grade. After the volunteers were pre-tested multiple concentrations, an evaluation table corresponding to each fishy taste degree was determined, as shown in Table 6.

TABLE 6
Evaluation of fish note in tasting

| | | Grade given | Desirable range |
|---|---|---|---|
| NO. | Description of fish note | I value | of I value |
| 1 | No fishy taste | I | (0, 1.0) |
| 2 | Slight fishy taste | II | (1, 0.2.0) |
| 3 | Acceptable fishy taste | III | (2, 0.3.0) |
| 4 | Very fishy, but still tolerable | VI | (3, 0.4.0) |
| 5 | Unacceptable fishy taste | V | (4, 0.5.0) |

A taste evaluation was performed of the four groups in EXAMPLE 4. None of the four groups was added with flavoring agent, and a single-blind test method was used. 30 volunteers with no knowledge of the composition of each group were asked to evaluate each group according to the evaluation table in Table 6, then take the average.

In addition, the group with the lowest I value among the above four groups of formula is flavored into five groups of soy milk flavor, hazelnut flavor, grapefruit cranberry flavor, cranberry flavor, and grapefruit flavor. 30 volunteers were asked to evaluate the fishy taste according to the evaluation table in Table 6, then take the average.

Result: As shown in Table 7, the fishy taste sensation of the four groups without adding flavoring agent. The I values grade of all 4 groups were lower than grade III, but the I value of group III was the lowest.

TABLE 7
Fishy taste grades of the four groups of lipid
emulsions without added flavoring agent

| Group | I value Grade | I value |
|---|---|---|
| Group I | III | 2.79 |
| Group II | III | 2.36 |
| Group III | II | 1.21 |
| Group VI | II | 1.68 |

Add different flavoring agents to Group III lipid emulsion. Based on the presence of flavoring agents, evaluate the fishy taste of group III lipid emulsions, and the taste used in the final formula has been determined. The results are shown in Table 8.

TABLE 8
Fishy taste grades of Group III with different flavoring agents

| Group | Taste | I value Grade | I value |
|---|---|---|---|
| 1 | Soy milk flavor | I | 0.03 |
| 2 | Hazelnut flavor | I | 0.06 |
| 3 | Grapefruit cranberry flavor | I | 0.04 |
| 4 | Cranberry flavor | I | 0.08 |
| 5 | Grapefruit flavor | I | 1.0 |

Combining the results of Embodiment 4 and 5, the inventors decided to use the fatty acid composition emulsion of Group III and added soy milk flavor and grapefruit cranberry flavor as products to the market. Human tests were performed before marketing to prove whether the product can quickly enrich Omega-3 fatty acids on the cell membranes of human tissues and organs.

### Example 7

### Rapid Enrichment of Omega-3 Fatty Acids on Cell Membranes in Human Tests

Next, the enrichment of EPA and DHA of the lipid emulsion on leukocyte phospholipid membrane, platelet phospholipid membrane of human was investigated. The lipid emulsion was added with grapefruit and cranberry flavoring agents to obtain 70% of Omega-3 fatty acids and 30% of MCT as a weight percent of total lipid.

43 volunteers were recruited; their Omega-3 index all less than 4%, and a BMI Mean±STD 26.94±2.93. The lipid emulsion was taken continuously for 28 days, and the daily dose was 4 g (calculated based on the weight of EPA+DHA). On day 0, day 2, day 4, day 7, 10, 14, 21, and 28, blood samples were taken to determine the EPA and DHA content on the leukocyte phospholipid membrane, platelet phospholipid membrane. The EPA and DHA content can be determined by GC-MS.

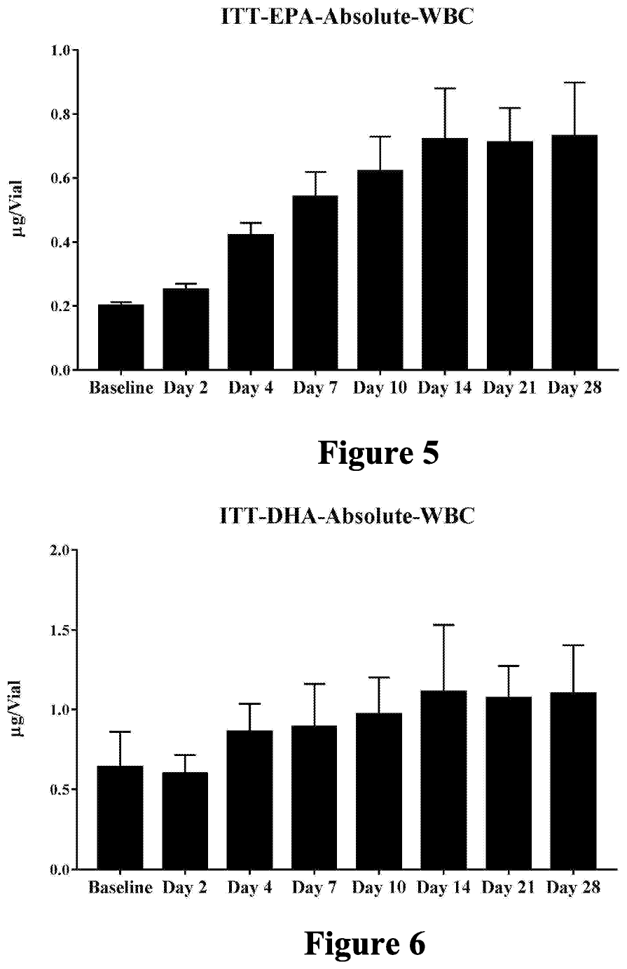

As shown in FIG. 5, on day 2 after taking in the lipid emulsion, the EPA enrichment on the leukocyte membrane of 43 volunteers increased by 20% compared with day 0, on day 4 increased by 105% compared with day 0, on day 7 increased by 165% compared with day 0, on day 10 increased by 205% compared with day 0, on day 14 increased by 255% compared with day 0, on day 21 increased by 250% compared with day 0, and on day 28 increased by 260% compared with day 0. It can be seen that after taking in the lipid emulsion for 15.65 days, the EPA on the leukocyte phospholipid membrane has reached the maximum enrichment level and after that, the enrichment level did not increase any substantial value.

As shown in FIG. 6, on day 2 after taking in the lipid emulsion, the DHA enrichment on the leukocyte membrane of 43 volunteers changed by nearly −9% compared with day 0, on day 4 increased by 30% compared with day 0, on day 7 increased by 34% compared with day 0, on day 10 increased by 47% compared with day 0, on day 14 increased by 70% compared with day 0, on day 21 increased by 63% compared with day 0, and on day 28 increased by 67% compared with day 0. It can be seen that after taking in the lipid emulsion for 13.86 days, the DHA on the leukocyte phospholipid membrane has reached the maximum enrichment level, subsequent increases in enrichment were modest.

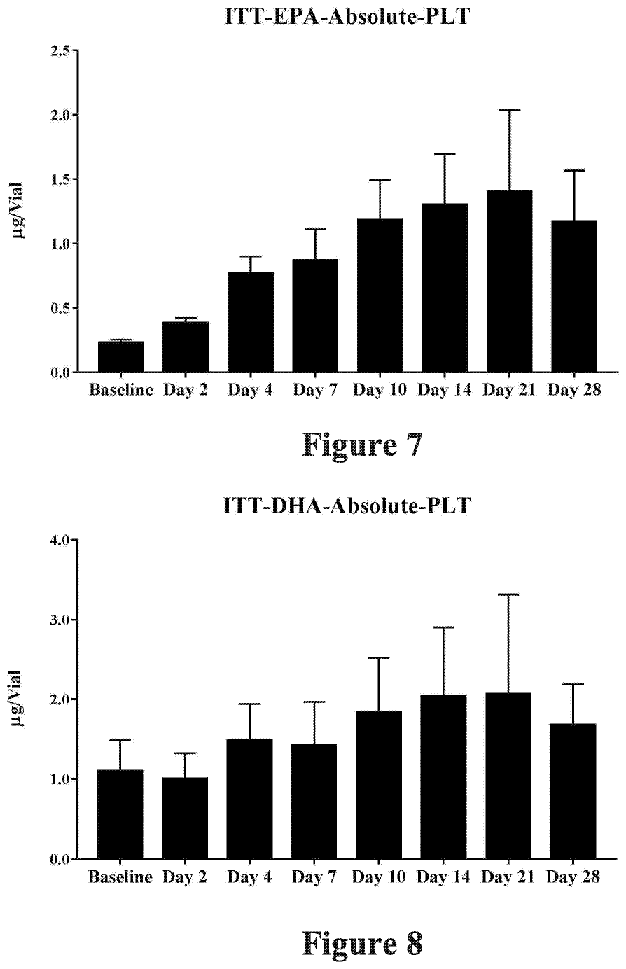

As shown in FIG. 7, on day 2 after taking in the lipid emulsion, the EPA enrichment on the platelet phospholipid membrane of 43 volunteers increased by 65% compared with day 0, on day 4 increased by 234% compared with day 0, on day 7 increased by 278% compared with day 0, on day 10 increased by 409% compared with day 0, on day 14 increased by 465% compared with day 0, on day 21 increased by 509% compared with day 0, and on day 28 increased by 409% compared with day 0. It can be seen that after taking in the lipid emulsion for 15.79 days, the EPA on the platelet phospholipid membrane has reached the maximum enrichment level, and after that, the enrichment level has not increased to any substantial value.

As shown in FIG. 8, on day 2 after taking in the lipid emulsion, the DHA enrichment on the platelet phospholipid membrane of 43 volunteers increased by −10% compared with day 0, on day 4 increased by 33% compared with day 0, on day 7 increased by 25% compared with day 0, on day 10 increased by nearly 77% compared with day 0, on day 14 increased by nearly 84% compared with day 0, on day 21 increased by 86% compared with day 0, and on day 28 increased by nearly 47% compared with day 0. It can be seen that after taking in the lipid emulsion for 13.79 days, the DHA on the platelet phospholipid membrane has reached the maximum enrichment level, and after that, the enrichment level has not increased to any substantial value.

As demonstrated in this disclosure, the lipid emulsion of the present invention can be administered enterally and can rapidly enrich Omega-3 fatty acids on the tissue and organ cell membranes of the subject, such as a person with Omega-3 fatty acid deficiency.

1. An isotonic lipid emulsion, comprising:
(i) 1% to 78% by weight of medium-chain triglycerides (MCT) based on the total amount of lipid within the emulsion, and
(ii) 22% to 99% by weight of fish oil or krill oil based on the total amount of lipid within the emulsion, wherein the fish oil is selected from the group consisting of natural fish oil, processed fish oil, purified fish oil concentrate, (re)esterified synthetic fish oil and mixtures thereof, and fish oil extracted from biologically-engineered microorganisms,
wherein the average particle size of emulsion particles is between about 10 nm and about 250 nm.

2. The isotonic lipid emulsion of claim 1, wherein the isotonic lipid emulsion is formulated for enteral administration.

3. The isotonic lipid emulsion of claim 1, wherein the isotonic lipid emulsion is formulated for enteral administration through an oral, nasal, or jejunal feeding tube.

4. The isotonic lipid emulsion of claim 1, wherein the average particle size of the emulsion particles is between about 10 nm and about 200 nm.

5. The isotonic lipid emulsion of claim 1, wherein at least 50% of the emulsion particles have particle sizes of 230 nm or less.

6. The isotonic lipid emulsion of claim 1, wherein at least 90% of the emulsion particles have particle sizes of 600 nm or less.

7. The isotonic lipid emulsion of claim 1, wherein the isotonic lipid emulsion is an oil-in-water emulsion.

8. The isotonic lipid emulsion of claim 7, wherein the oil component in the isotonic lipid emulsion has a concentration of from about 2 g/100 mL to about 20 g/100 mL.

9. The isotonic lipid emulsion of claim 1, wherein the isotonic lipid emulsion has a pH between about 3.0 and about 8.0.

10. The isotonic lipid emulsion of claim 9, wherein the isotonic lipid emulsion has a pH between about 3.0 and about 4.5 when the F0 value of sterilization temperature and time is less than or equal to 1.

11. The isotonic lipid emulsion of claim 9, wherein the isotonic lipid emulsion has a pH between about 6.5 and about 7.5 when the F0 value of sterilization temperature and time is greater than or equal to 8.

12. The isotonic lipid emulsion of claim 9, wherein the osmotic pressure of the isotonic lipid emulsion is between about 280 mmol/L and about 320 mmol/L.

13. (canceled)

14. The isotonic lipid emulsion of claim 1, wherein total lipid content is between about 2% and about 60% by weight of the liquid emulsion.

15. (canceled)

16. The isotonic lipid emulsion of claim 1, wherein the MCT contains 6 to 14 carbon atoms.

17. The isotonic lipid emulsion of claim 1, wherein the MCT contains at least about 90% caprylic acid (C8), capric acid (C10), or a combination thereof.

18. (canceled)

19. The isotonic lipid emulsion of claim 1, wherein the fish oil is based on fatty acid methyl ester of fish oil concentrate and contains between about 25% and about 95% by weight of eicosapentaenoic acid (EPA) based on the total weight of the fish oil.

20. The isotonic lipid emulsion of claim 1, wherein the fish oil contains between about 12% and about 95% by weight of docosahexaenoic acid (DHA) based on the total weight of the fish oil.

21. (canceled)

22. A composition or a food or drink comprising the isotonic lipid emulsion of claim 1.

23. (canceled)

24. (canceled)

25. (canceled)

26. A method of administering a lipid emulsion, comprising administering enterally a dose of the isotonic lipid emulsion of claim 1, to a subject in need thereof.

27. (canceled)

28. (canceled)

29. A method for treating a human having Omega-3 fatty acid deficiency, comprising administering enterally to the human an effective dosage amount of the isotonic lipid emulsion of claim 1, thereby increasing enrichment of Omega-3 fatty acids on cell membrane of human tissue and organ by at least 10% compared to a predetermined reference value.

30. (canceled)

31. (canceled)

32. (canceled)

33. (canceled)

34. (canceled)
